(19) United States
(12) Patent Application Publication
(10) Pub. No.: US 2024/0006074 A1
(43) Pub. Date: Jan. 4, 2024

(54) HEALTH SCORE GENERATION ON MEDICAL DEVICE

(71) Applicant: Welch Allyn, Inc., Skaneateles Falls, NY (US)

(72) Inventors: Thomas A. Myers, Syracuse, NY (US); Tiffany Do, Milwaukee, WI (US); Catherine M. King, Fayetteville, NY (US); Christopher L. Long, Chittenango, NY (US); Tiffany L. Moon, Skaneateles Falls, NY (US); Rebecca Quilty-Koval, Baldwinsville, NY (US); Ching Yue Yeung, Skaneateles Falls, NY (US)

(21) Appl. No.: 18/338,788

(22) Filed: Jun. 21, 2023

Related U.S. Application Data
(60) Provisional application No. 63/367,495, filed on Jul. 1, 2022.

(51) Int. Cl. G16H 50/30; G16H 10/60; G16H 50/70

(52) U.S. Cl. CPC G16H 50/30; G16H 10/60; G16H 50/70

(57) ABSTRACT

A medical device for assessing a health status of a patient is described. The medical device receives a selection of two or more health scores, and obtains physiological parameter measurements from one or more sensor modules based on the selection of the two or more health scores. The medical device generates the two or more health scores using the physiological parameter measurements received from the one or more sensor modules. The two or more health scores are generated based on a single workflow for assessing the patient. The medical device displays the two or more health scores on a display screen.

### BACKGROUND

Caregivers typically use various types of healthcare equipment to provide healthcare to a patient in a clinical care environment. The healthcare equipment can include multifunction medical devices that perform more than one function during patient assessment. Such multifunction medical devices are used to measure one or more physiological parameters from a single patient or from multiple patients within the clinical care environment. Also, such multifunction medical devices typically include various sensor modules and peripheral components for measuring the one or more physiological parameters.

A health score, such as an early warning score, is a tool used by caregivers to quickly determine a degree of patient illness. Multiple types of health scores are typically used within a clinical care environment. These types of scores are determined based on different combinations of physiological parameters and clinical observations.

### SUMMARY

In general terms, the present disclosure relates to a medical device for assessing a health status of a patient based on two or more health scores. In one possible configuration, the medical device provides a technical effect by generating the two or more health scores based on a single workflow for assessing the patient. Various aspects are described in this disclosure, which include, but are not limited to, the following aspects.

One aspect relates to a medical device for assessing a health status of a patient, the medical device comprising: at least one processing device; and a memory device storing instructions which, when executed by the at least one processing device, cause the at least one processing device to: receive a selection of two or more health scores; obtain physiological parameter measurements from one or more sensor modules based on the selection of the two or more health scores; generate the two or more health scores using the physiological parameter measurements received from the one or more sensor modules, the two or more health scores being generated based on a single workflow for assessing the patient; and display the two or more health scores on a display screen.

Another aspect relates to a medical device for assessing a health status of a patient, the medical device comprising: at least one processing device; and a memory device storing instructions which, when executed by the at least one processing device, cause the at least one processing device to: determine a clinical setting of the medical device; filter a plurality of health scores based the clinical setting; display a subset of health scores from the plurality of health scores, the subset of health scores including health scores determined appropriate for the clinical setting; receive a selection of two or more health scores from the subset of health scores; obtain physiological parameter measurements based on the selection of the two or more health scores; generate the two or more health scores using the physiological parameter measurements; and store the two or more health scores in an electronic medical record.

Another aspect relates to a method of assessing a patient in a clinical care environment, the method comprising: determining a clinical setting under which the medical device is being operated; filtering a plurality of health scores based the clinical setting; displaying a subset of health scores from the plurality of health scores, the subset of health scores including health scores determined appropriate for the clinical setting; receiving a selection of two or more health scores from the subset of health scores; obtaining physiological parameter measurements based on the selection of the two or more health scores; generating the two or more health scores using the physiological parameter measurements; and storing the two or more health scores in an electronic medical record.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for collecting physiological parameter measurements from patients in a clinical care environment.

FIG. 2 illustrates an example of a medical device of the system of FIG. 1.

FIG. 3 schematically illustrates an example of a method of generating health scores on the medical device of FIG. 2.

FIG. 4 illustrates an example of a user interface generated on the medical device of FIG. 2, the user interface including multiple health scores.

FIG. 5 illustrates another example of a user interface generated on the medical device of FIG. 2, the user interface including multiple health scores.

FIG. 6 schematically illustrates an example of a health score defined for use in the clinical care environment of FIG. 1.

FIG. 7 schematically illustrates an example of a method of displaying one or more health scores for selection on the medical device of FIG. 2.

FIG. 8 schematically illustrates in more detail an example of an operation of determining a clinical setting in the method of FIG. 7.

FIG. 9 schematically illustrates an example of a method of displaying one or more previously generated health scores on the medical device of FIG. 2.

FIG. 10 illustrates an example of a user interface generated on the medical device of FIG. 2, the user interface including a trend of previously generated health scores.

FIG. 11 schematically illustrates an example of a method of generating on the medical device of FIG. 2 one or more health scores based on one or more disease states.

FIG. 12 illustrates an example of a user interface generated on the medical device of FIG. 2, the user interface includes one or more health scores having one or more disease variants.

FIG. 13 schematically illustrates an example of the medical device of FIG. 2.

### DETAILED DESCRIPTION

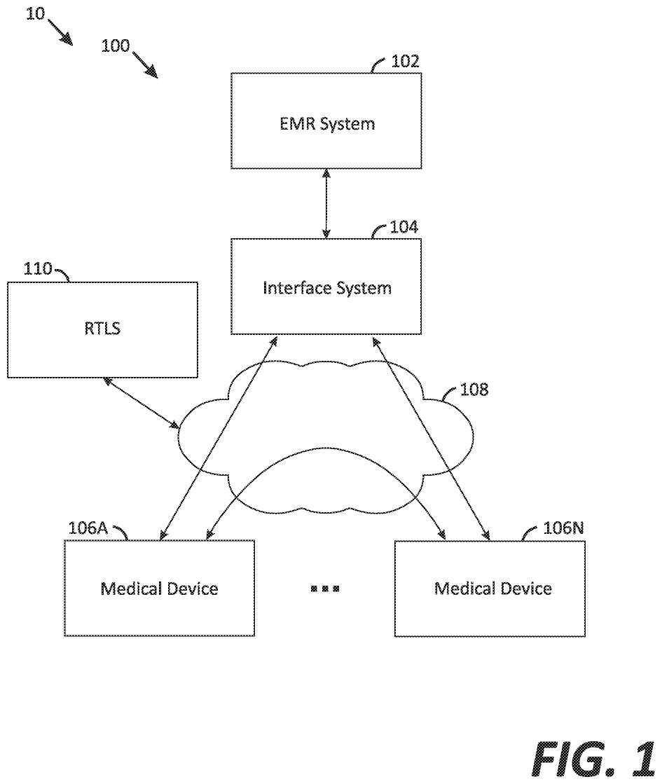

FIG. 1 schematically illustrates an example of a system 100 for collecting physiological parameter measurements from patients in a clinical care environment 10. An example of the clinical care environment can include a healthcare facility such as a hospital, a surgical center, a nursing home, a long term care facility, or similar type of facility. As shown in FIG. 1, the system 100 includes an Electronic Medical Records (EMR) system 102, an interface system 104, medical devices 106A-106N, and a network 108.

The network 108 is a communications network that facilitates data communication between the medical devices 106A-106N, and between the medical devices 106A-106N and the interface system 104. The network 108 can include a set of computing devices and links between the computing devices. The computing devices in the network 108 use the links to enable data communication among the computing devices in the network. The network 108 can include routers, switches, mobile access points, bridges, hubs, intrusion detection devices, storage devices, standalone server devices, blade server devices, sensors, desktop computers, firewall devices, laptop computers, tablet computers, handheld computers, smartphones, and other types of computing devices. In various embodiments, the network 108 includes various types of links. For example, the network 108 can include wired and/or wireless links. In some embodiments, the network 108 is implemented at various scales. For example, the network 108 can be implemented as one or more local area networks (LANs), metropolitan area networks, subnets, wide area networks (such as the Internet), or can be implemented at another scale.

The EMR system 102 is a computing system that allows storage, retrieval, and manipulation of electronic medical records. As used herein, a computing system is a system of one or more computing devices. A computing device is a physical, tangible device that processes data. Example types of computing devices include personal computers, standalone server computers, blade server computers, mainframe computers, laptop computers, tablet computers, handheld computers, smartphones, and other types of electronic devices that process data.

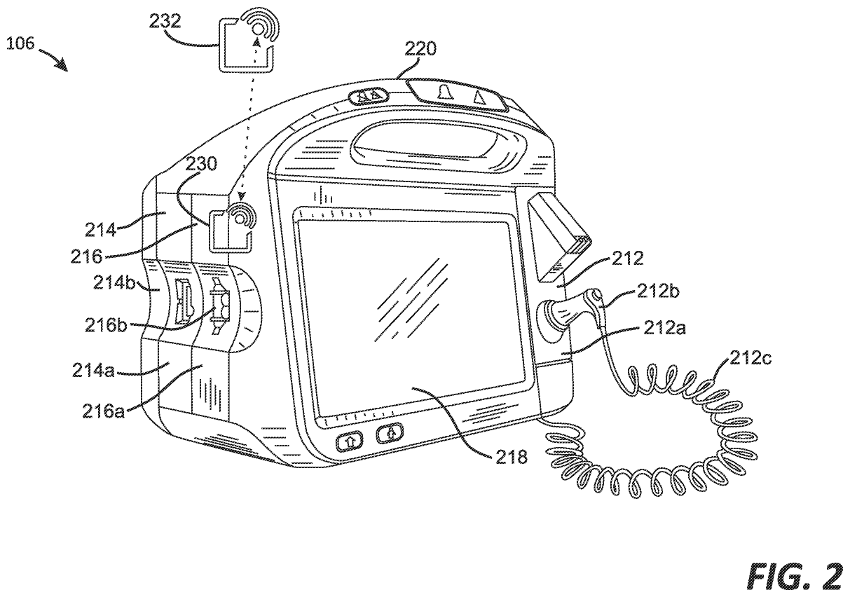

Each of the medical devices 106 is a computing device. The medical devices 106 can provide various types of functionalities. For example, the medical devices 106 can include a physiological parameter monitoring platform or spot monitor, such as the one illustrated in FIG. 2. In such examples, the physiological parameter monitoring platform or spot monitor can be used by a clinician to measure and/or monitor physiological parameters of multiple patients, and to display representations of the measured physiological parameters.

In further examples, the medical devices 106 can include any type of physical assessment device such as, without limitation, ophthalmoscopes, otoscopes, dermatoscopes, vision screeners, and the like. The medical devices 106 can further include other types of devices that are capable of measuring physiological parameters such as hospital beds. The medical devices 106 can communicate with each other through the network 108.

The interface system 104 is a computing system that acts as an interface between the EMR system 102 and the medical devices 106. In some embodiments, the interface system 104 includes Connex® Vitals Management Software from Hillrom of Batesville, Indiana.

As an illustrative example, the interface system 104 provides a single software interface for each of the medical devices 106 such that the medical devices can send requests to the software interface provided by the interface system 104. When the interface system 104 receives a request from one of the medical devices 106, the interface system 104 translates the request into a request that works with the software interface provided by the EMR system 102. The interface system 104 then provides the translated request to the EMR system 102. When the interface system 104 receives a response from the EMR system 102, the interface system 104 translates the response into a format understood by the medical devices 106, and then forwards the translated response to an appropriate one of the medical devices 106. In this manner, the interface system 104 allows use of the medical devices 106 in various types of EMR systems.

The medical devices 106 can send various types of data to the interface system 104 for storage in the EMR system 102 and can receive various types of data from the EMR system 102 through the interface system 104. For example, a medical device 106 can send physiological parameter measurements and clinical observations to the interface system 104 for storage in the EMR system 102. In further examples, a medical device 106 can retrieve past physiological parameter measurements, clinical observations, lab results, scans, and other patient health information from the EMR system 102 through the interface system 104.

As further shown in FIG. 1, a real-time locating system (RTLS) 110 is connected to the network 108. The RTLS 110 will be described in more detail below with reference to FIG. 2.

FIG. 2 illustrates an example of a medical device 106 of the system 100. In this example, the medical device 106 is a physiological parameter monitoring platform or spot monitor. The medical device 106 can include one or more sensor modules. Each of the sensor modules is configured to measure one or more physiological parameters of a patient.

In the illustrative example shown in FIG. 2, the medical device 106 includes a temperature sensor module 212 that is accessible from a front side of the device, and a photoplethysmogram sensor module 214 and a non-invasive blood pressure (NIBP) sensor measurement module 216 that are accessible from a left hand side of the device. As used herein, a “module” is a combination of physical structure which resides in the medical device 106 and peripheral components that attach to and reside outside of the medical device 106. The medical device 106 can include additional sensor modules for receiving additional physiological parameter measurements, including ECG or EKG measurements.

The temperature sensor module 212 is designed to receive body temperature measurements of a patient. As an illustrative example, the temperature sensor module 212 includes a front panel 212a that has an outer surface accessible from the front side of the medical device 106. The front panel 212a provides access to a wall (not shown) storing a removable probe (not shown), also referred to as a temperature probe, which is attached to a probe handle 212b. The temperature probe and the probe handle 212b are tethered to the temperature sensor module 212 via an insulated conductor 212c. The temperature probe is designed to make physical contact with the patient in order to sense the body temperature of the patient.

The photoplethysmogram sensor module 214 can be used to measure blood oxygen saturation and pulse. Also, the photoplethysmogram sensor module 214 can be used to measure respiration rate based on changes in a plethysmography waveform. The photoplethysmogram sensor module 214 includes a front panel 214a having a connector port 214b that enables a connection between a pulse oximeter (not shown) and the photoplethysmogram sensor module 214 residing inside the medical device 106. The pulse oximeter resides externally, and is configured to interoperate with the photoplethysmogram sensor module 214 when connected to the photoplethysmogram sensor module 214 via the connector port 214b. The pulse oximeter can include a clip that attaches to an appendage (e.g., finger) of the patient. The clip includes an infrared light transmitter, and a sensor that detects and measures blood oxygen saturation and pulse rate based on transmission of the infrared light through the patient's appendage.

The NIBP sensor module 216 can be used to measure blood pressure of the patient. The NIBP sensor module 216 includes a front panel 216a having a connector port 216b that enables a connection between one or more peripheral NIBP components and the NIBP sensor module 216 residing inside the medical device 106. The peripheral NIBP components reside externally, and are configured to interoperate with the NIBP sensor module 216 when connected to the NIBP sensor module 216 via the connector port 216b. The peripheral NIBP components can include an inflatable cuff that attaches to an appendage of the patient, such as an upper arm. The inflatable cuff is used to measure systolic and diastolic blood pressure of the patient, mean arterial pressure (MAP) of the patient, and pulse rate of blood flowing within the patient.

As shown in FIG. 2, a front side of the medical device 106 includes a display screen 218 that can display graphical representations of the physiological parameter measurements received from the various sensor modules of the medical device 106 including the temperature sensor module 212, the photoplethysmogram sensor module 214, and the NIBP sensor module 216. The display screen 218 can further display graphical representations of additional physiological parameter measurements and clinical observations from additional sources such as from other medical devices and the EMR system 102 via connections through the network 108. In some examples, the display screen 218 is a touchscreen that receives manual inputs from a user of the medical device 106. Also, the medical device 106 can include one or more handles 220 on its housing that enable the medical device 106 to be carried by hand by the user.

The medical device 106 is able to operate within one or more workflows. A workflow is a series of one or more tasks that a user of the medical device 106 performs. When the medical device 106 operates within a workflow, the medical device 106 provides functionality suitable for assisting the user (e.g., caregiver) in performing the workflow. When the medical device 106 operates within different workflows, the medical device 106 provides different functionality.

When the medical device 106 is manufactured, the medical device 106 is configured to be able to operate within one or more predefined workflows. After the medical device 106 is manufactured, the medical device 106 can be reconfigured to operate within one or more additional and/or customized workflows. In this way, the clinical care environment 10 (such as a hospital) can adapt the medical device 106 for use in customized workflows as desired.

As an illustrative example, the medical device 106 can operate in a monitoring workflow or in a non-monitoring workflow. A monitoring workflow can include continuously monitoring the physiological parameters of a single patient. Example types of non-monitoring workflows include a spot check workflow and/or a triage workflow.

When the medical device 106 operates in the monitoring workflow, the medical device 106 obtains a series of measurements of one or more physiological parameters (e.g., temperature, SpO2, blood pressure, EKG, and the like) of a single patient over a period of time. In addition, the medical device 106 displays, on the display screen 218, a monitoring workflow home screen. The monitoring workflow home screen can include a representation of the physiological parameters measured from the patient who is being monitored.

As an illustrative example, when the medical device 106 operates in the monitoring workflow, the medical device 106 can obtain a blood pressure measurement of a single patient once every ten minutes for six hours. The medical device 106 can display a monitoring workflow home screen on the display screen 218 that displays a representation of the blood pressure measurements measured from the patient. In this way, the medical device 106 allows a user such as caregiver in the healthcare facility to monitor a health status of the patient.

When the medical device 106 operates within a non-monitoring workflow, the medical device 106 obtains measurements of one or more physiological parameters from a series of patients that have been previously identified. As used herein, a patient is previously identified when the medical device 106 stores information regarding the identity of the patient. The medical device 106 can display a non-monitoring workflow home screen on the display screen 218 that displays a representation of a physiological parameter measured from a previously identified patient in the series of patients. In this way, the medical device 106 allows a user such as caregiver in the healthcare facility to check a health status of multiple patients.

In another example, when the medical device 106 operates in a triage workflow, the medical device 106 can obtain measurements of physiological parameters from each patient in a series of unidentified patients, such as when the patients arrive in an emergency department of a hospital. In this example, the medical device 106 displays a triage workflow home screen that displays a representation of a physiological parameter measured from a patient who has not been previously identified. In this way, the medical device 106 can perform triage on a series of unidentified patients as they arrive. As used herein, a patient is an unidentified patient when the medical device 106 does not store information regarding the identity of the patient.

The monitoring workflow home screen can be different from the non-monitoring workflow home screen in various ways. For example, the monitoring workflow home screen can include at least one user-selectable control that is not included in the non-monitoring workflow home screen and/or the non-monitoring workflow home screen can include at least one user-selectable control that is not included in the monitoring workflow home screen. As another example, different navigation options can be provided in the different workflows to allow for more efficient navigation within the respective workflows based on their particular functions and/or needs. Additional examples of the differences between the monitoring workflow home screen and the non-monitoring workflow home screen are contemplated.

As further shown in the illustrative example provide in FIG. 2, a tag 230 is attached to or otherwise included on the medical device 106. In this example, the tag 230 communicates with fixed reference points 232 that are positioned in designated locations throughout the clinical care environment 10. The communication between the tags 230 and the fixed reference points 232 is accomplished through wireless signals such as through radio frequency (RF), optical (e.g., infrared), or acoustic (e.g., ultrasound) communications technologies. The tag 230 and the fixed reference points 232 can be transmitters, receivers, or both.

In some examples, the tag 230 and fixed reference points 232 are used by the RTLS 110 to automatically identify and track the location of the medical device 106 within the clinical care environment 10 in real-time. For example, a fixed reference point 232 can receive wireless signals from the tag 230, and can communicate the wireless signals to the RTLS 110 via the network 108. In such example, the RTLS 110 determines based on the wireless signals received by the fixed reference point 232 from the tag 230 that the medical device 106 is in the designated location of the fixed reference point 232. In alternative examples, the tag 230 can receive wireless signals from a fixed reference point 232, and the medical device 106 can use the wireless signals to determine its location within the clinical care environment 10.

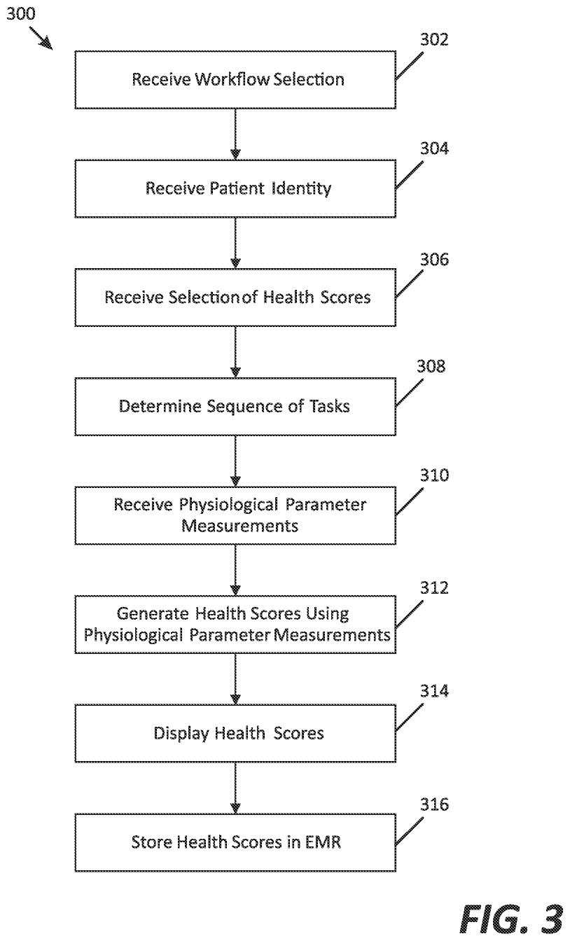

FIG. 3 schematically illustrates an example of a method 300 of generating health scores on the medical device 106. The health scores are composite scores that are based on multiple physiological parameters and/or clinical observations. The health scores can be used by a user of the medical device 106, such as a caregiver that is providing healthcare to a patient, to assess a health status of the patient. For example, the health scores are indicative of a degree of patient illness or a likelihood that the patient will experience an adverse medical event such as cardiac arrest, a patient fall, or sepsis. The health scores generated by the medical device 106 can include protocols such as instructions to perform certain medical interventions defined for use in the clinical care environment 10 to avoid severe health deterioration of the patient.

The health scores are calculated based on different combinations of physiological parameter measurements and/or clinical observations received by the various sensor modules of the medical device 106. Examples of the physiological parameter measurements that can be used to calculate the health scores can include, without limitation, respiration rate, blood oxygen saturation, body temperature, blood pressure, and pulse/heart rate. Certain health scores may use at least in part overlapping physiological parameter measurements.

In accordance with the examples described above, the body temperature of a patient can be acquired from the temperature sensor module 212, the pulse, blood oxygen saturation, and respiration rate of the patient can be acquired from the photoplethysmogram sensor module 214, and the blood pressure of the patient can be acquired from the NIBP sensor module 216. The health scores can also be based on one or more clinical observations of a patient such as an alert, verbal, pain, unresponsive (AVPU) score, which can be manually entered by a user of the medical device 106, such as a caregiver or nurse who provides healthcare to the patient.

Each physiological parameter measurement and clinical observation is compared to a normal or healthy range, and is allocated an individual parameter sub-score. An individual parameter sub-score of 0 indicates that the physiological parameter measurement or clinical observation is within the normal or healthy range, and individual parameter sub-scores greater that 0 indicate that the physiological parameter measurement or clinical observation is outside of the normal or healthy range. The individual parameter sub-scores increase in numerical value as they move farther away from the normal or healthy range. The individual parameter sub-scores are added together to compute the health score, and the health score can then be compared to a threshold to determine whether to trigger an alarm and/or to perform a medical intervention or protocol due to a likelihood of severe health deterioration and/or an adverse medical event.

As used herein, the health scores can include the Early Warning Score (EWS), and any variations thereof including the National Early Warning Score (NEWS & NEWS2), Modified Early Warning Score (MEWS), Modified Early Obstetric Warning Score (MEOWS), Pediatric Early Warning Score (PEWS). Additionally, the health scores can include the Quick Sequential Organ Failure Assessment Score (QSOFA) for determining a likelihood of developing sepsis, the Glasgow Coma Scale (GCS) for determining level of consciousness, and the Morse Fall Scale for determining a patient's likelihood of falling.

Additionally, the health scores can include scores that are specially defined and/or customized for use within the clinical care environment 10. For example, the clinical care environment 10 can define customized health scores by modifying the types of physiological parameter measurements and clinical observation used to calculate a health score, the normal or healthy ranges used for determining the individual parameter sub-scores, and/or the weights that are assigned to the individual parameter sub-scores for calculating the health score.

Referring now to FIG. 3, the method 300 can include an operation 302 of receiving a selection of a workflow by a user of the medical device. Operation 302 can include displaying workflows for selection by the user of the medical device 106 including a monitoring workflow, a spot check workflow, and a triage workflow in accordance with the examples described above. Operation 302 can further include displaying additional workflows for selection by the user that are specially defined and/or customized for use within the clinical care environment 10.

Next, the method 300 can include an operation 304 of receiving a patient identity. Operation 304 can include receiving the patient identity from a module on the medical device 106 that includes a scanner for use by a user of the medical device 106 to scan a barcode on a wristband or other accessory worn by the patient to identify the patient. Alternatively, operation 304 can include receiving a selection of the patient from a menu displayed on the display screen 218. Additional techniques for receiving the patient's identity are possible.

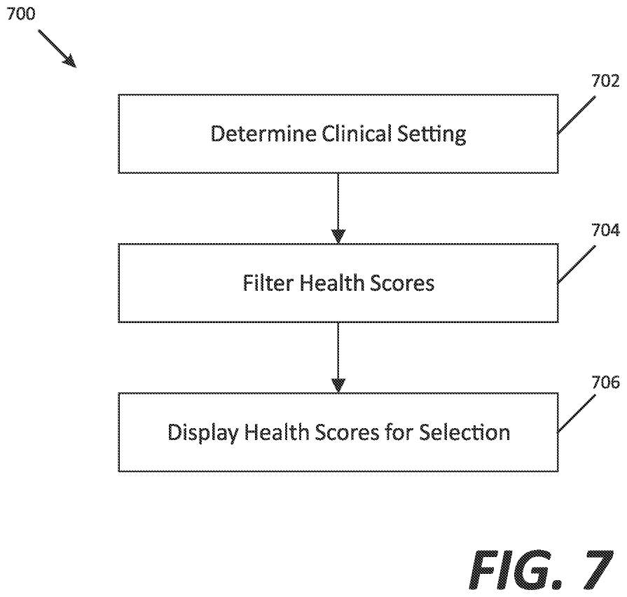

Next, the method 300 includes an operation 306 of receiving a selection of health scores by the user of the medical device 106. For example, a plurality of different health scores including, without limitation, EWS, NEWS, NEWS2, MEWS, MEOWS, PEWS, QSOFA, Morse Fall Scale, and/or a plurality of customized health scores that are specially defined for use within the clinical care environment 10 are displayed on the display screen 218 for selection by a user of the medical device 106. In some examples, the plurality of different health scores is displayed on the display screen 218 for selection by a user of the medical device 106 in accordance with a method 700, which will be described in more detail below with reference to FIG. 7. Operation 306 can include receiving a selection of two or more health scores, such as when it is desirable to generate more than one health score during a single patient encounter.

Next, the method 300 can include an operation 308 of determining a sequence of tasks for the user of the medical device 106 to perform based on at least the selection of health scores received in operation 306. For example, the sequence of tasks can include tasks to collect physiological parameter measurements and clinical observation needed for the calculation of the health score selections received in operation 306. In some examples, the sequence of tasks can also be based on the workflow selection received in operation 302 and/or the patient identity received in operation 304. For example, the sequence of tasks determined in operation 308 may differ depending on whether a monitoring workflow or a non-monitoring workflow is selected in operation 302. As another example, the sequence of tasks determined in operation 308 may differ depending on whether the patient is diagnosed with a certain disease state or not.

Next, the method 300 includes an operation 310 of receiving physiological parameter measurements from the patient as the user of the medical device 106 performs the sequence of tasks determined in operation 308. Operation 310 can include receiving one or more physiological parameter measurements such as, without limitation, respiration rate, blood oxygen saturation, temperature, blood pressure, pulse/heart rate, as well as clinical observations such as the AVPU score. Operation 310 can include receiving additional types of physiological parameter measurements and observations, and different combinations thereof.

Next, the method 300 includes an operation 312 of generating health scores using the physiological parameter measurements and scores received in operation 310. Operation 312 can include simultaneously calculating two or more health scores when a selection of two or more health scores is received in operation 306. Thereafter, the method 300 can include an operation 314 of displaying the health scores on the display screen 218 of the medical device 106. In some further examples, the method 300 can include an operation 316 of storing the health scores in the electronic medical record of the patient in the EMR system 102 via the network 108.

Advantageously, the method 300 can eliminate the need for the user of the medical device 106 to recollect duplicate physiological parameter measurements and clinical observations for generating multiple health scores. Instead, the method 300 when performed on the medical device 106 allows the user to select multiple health scores before starting a patient assessment workflow such that multiple health scores are simultaneously generated by the medical device 106 based on a single workflow for assessing a patient by reusing overlapping physiological parameter measurements and clinical observations. This can reduce the encounter time that the caregiver spends with the patient to generate the multiple health scores, and can potentially improve patient comfort and satisfaction in the clinical care environment 10.

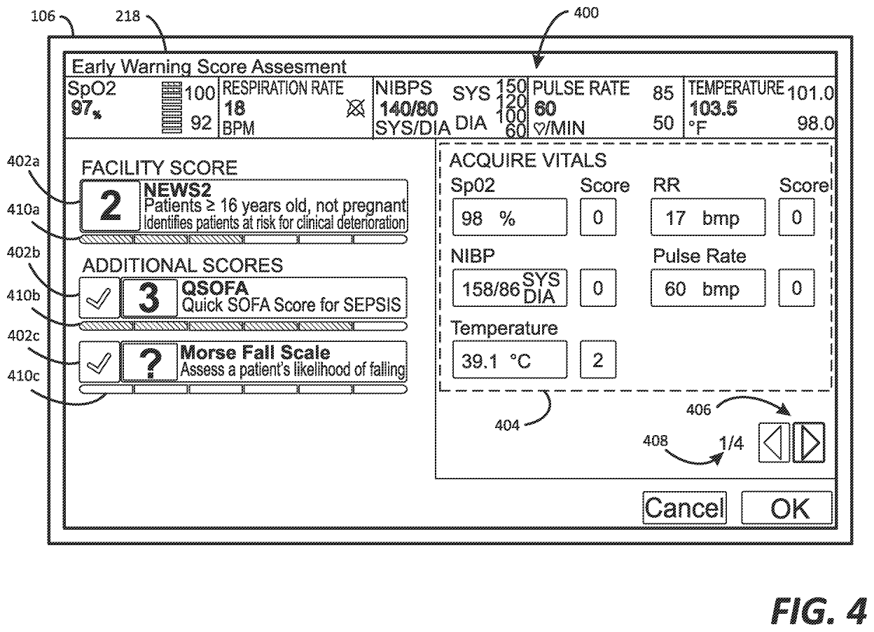

FIG. 4 illustrates an example of a user interface 400 that can be generated on the display screen 218 of the medical device 106. The user interface 400 includes health scores 402a-402c that are simultaneously generated based on the method 300.

In this illustrative example, the user interface 400 displays a first health score 402a, which includes a NEWS2 score designated as a “facility score”. The facility score can include one or more health scores that are preferred for use in the clinical care environment 10. In some examples, the facility scores are automatically generated each time a caregiver assesses a patient in the clinical care environment 10. The user interface 400 further includes “additional scores” such as a second health score 402b, which includes a QSOFA score, and a third health score 402c, which includes a Morse Fall Scale. The user interface 400 can display additional types of health scores based on the selection of health scores in operation 306 of the method 300.

The user interface 400 includes a screen 404 for entering and viewing physiological parameters and clinical observations for computing the health scores 402. The screen 404 displays an SpO2 measurement of 98% which is within a normal range such that it has a parameter sub-score of “0”, a non-invasive systolic/diastolic blood pressure of 158/86 which is within a normal range such that it has a parameter sub-score of “0”, a body temperature of 39.1° C. that is elevated and has a parameter sub-score of “2”, a respiration rate of 17 breaths per minute which is within a normal range such that it has a parameter sub-score of “0”, and a pulse rate of 60 beats per minute which is within a normal range such that it has a parameter sub-score of “0”. The parameter sub-scores are added together to calculate the health scores 402.

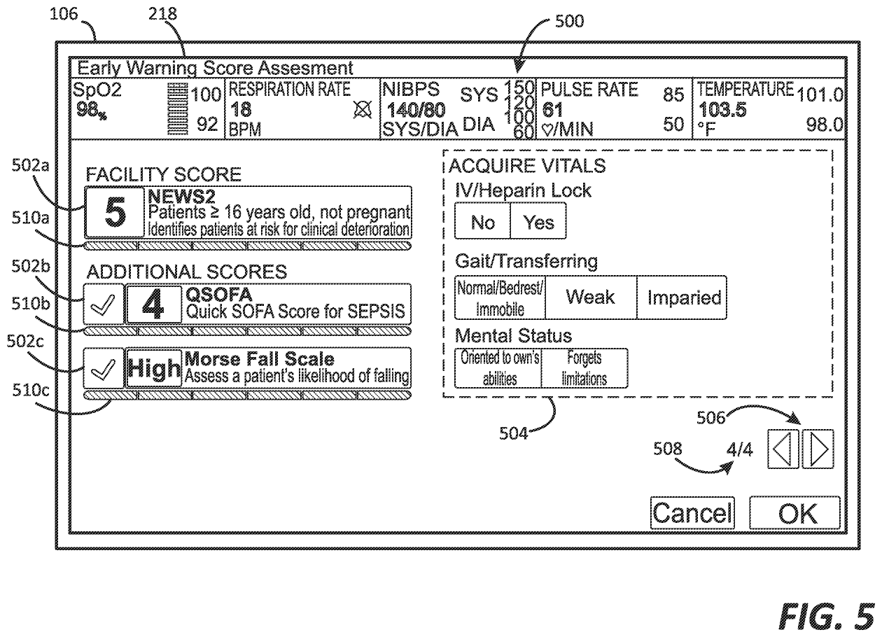

The user interface 400 further includes toggle inputs 406 that are selectable to move through pages of the screens 404 to enter and view additional physiological parameter measurements and clinical observations for calculation of the health scores 402. The user interface 400 displays an icon 408 next to the toggle inputs 406 to indicate the page number and total number of pages of the screen 404. Each page of the screen 404 displays additional physiological parameter measurements, clinical observations, and parameter sub-scores upon which the health scores 402 are based. Another example of a page of the screen 404 displaying physiological parameter measurements and parameter sub-scores is shown in FIG. 5.

The user interface 400 can further include individual progress indications 410 of data entries for each of the health scores 402. For example, each of the health scores 402a-402c can be computed based on different types of inputs such as physiological parameter measurements and clinical observations. As a user enters the inputs into the screen 404, the individual progress indications 410a-410c populate to indicate a state of completion for computing each of the health scores. In the example shown in FIG. 4, the individual progress indications 410a-410c each include six bars, and the first health score 402a has three of the six bars populated indicating that the inputs for computing the first health score 402a are incomplete. The second health score 402b has five of the six bars populated indicating that the inputs for computing the second health score 402b are incomplete. The third health score 402c does not have any of the six bars populated indicating that none of the inputs for computing the third health score 402c have been entered.

FIG. 5 illustrates another example of a user interface 500 that can be generated on the display screen 218 of the medical device 106. In this example, the user interface 500 includes health scores 502a-502c that can be generated based on performance of the method 300. In this example, a first health score 502a includes a NEWS2 score having a value of “5”, a second health score 502b includes a QSOFA score having a value of “4”, and a third health score 502c includes a Morse Fall Scale score having a value of “4”. Also, in this example, individual progress indications 510a-510c for each of the health scores 502a-502c have all six bars populated, indicating that the inputs for computing the health scores 502a-502c are complete.

In the example illustrated in FIG. 5, the health scores 502a-502c are highlighted to have a certain color to indicate their severity. For example, the health scores can be highlighted to have a green color to indicate a healthy score, yellow to indicate moderate score that should be monitored, and red to indicate a severe score indicating a high risk for patient deterioration and/or adverse health events. In this example, the first health score 502a is highlighted in yellow, while the second and third health scores 502b, 502c are each highlighted in red.

In the illustrative example provided in FIG. 5, the user interface 500 displays acquired physiological parameters 504 that include clinical observations upon which the third health score 502c (e.g., Morse Fall Scale) is based. Additionally, the user interface 500 further includes one or more toggle inputs 506, and an icon 508 to provide an indication of the page number and total number of pages of the acquired physiological parameters 504.

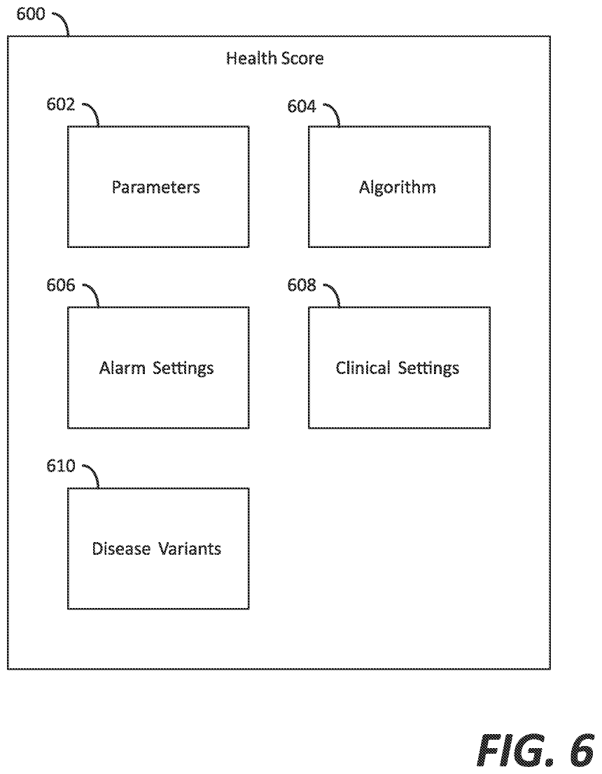

FIG. 6 schematically illustrates an example of a health score 600 that can be defined for use in the clinical care environment 10. The health score 600 is customizable. For example, the health score 600 can be defined based on a custom selection of physiological parameters 602, such as one or more of the physiological parameters and clinical observations described above.

The health score 600 further includes an algorithm 604 that defines how the health score 600 is computed based on the custom selection of the physiological parameters 602. For example, the algorithm 604 can assign different weights to the physiological parameters 602 to emphasize or deemphasize certain parameters. The algorithm can also include multipliers and other coefficients that are used for calculating the health score 600.

The health score 600 further includes alarm settings 606 that determines when the heath score triggers an alarm or medical protocols within the clinical care environment 10. The alarm settings 606 can define one or more thresholds or classifications. For example, when the health score 600 exceeds a threshold defined in the alarm settings 606, an alarm on the medical device 106 is triggered which can cause alerts and/or notifications to be generated and sent throughout the clinical care environment 10 for providing a medical intervention. As another example, when the health score 600 is within a certain classification defined in the alarm settings 606, a medical protocol can be displayed on the medical device 106 for a caregiver to perform one or more tasks to improve a condition of the patient, and avoid patient health deterioration.

The health score 600 further includes clinical settings 608 which can include settings that define appropriate uses of the health score 600. For example, the clinical settings 608 can prevent a selection of the health score 600 in operation 306 of the method 300 when it is determined that the health score 600 would be inappropriate for use in a context that the medical device 106 is being used. For example, the clinical settings 608 can allow selection of the health score 600 only when the medical device 106 is determined to be located within a certain department, clinic, or area of the clinical care environment 10, when the medical device 106 is being used in certain workflows in the clinical care environment 10, and/or when the medical device 106 is being used to monitor certain patients in the clinical care environment 10.

As an illustrative example, the clinical settings 608 allow selection of a Pediatric Early Warning Score (PEWS) in operation 306 of the method 300 only when the medical device 106 is determined to be located within a pediatric department within the clinical care environment 10, since it would be inappropriate to generate this health score in other departments or areas. As another illustrative example, the clinical settings 608 allow selection of the PEWS score in operation 306 of the method 300 only for patients who are identified in operation 304 as pediatric patients, since it would be inappropriate to select and generate this health score for other types of patients. Additional examples of the clinical settings 608 that can prevent the selection and subsequent generation of the health score 600 based on the context in which the medical device 106 is being operated by a user, are contemplated. Additionally, two or more different types of settings can be combined to limit the selection of the health score 600.

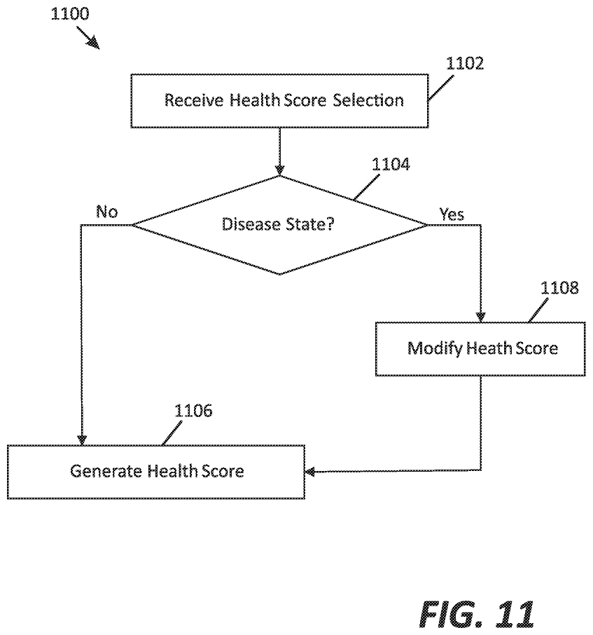

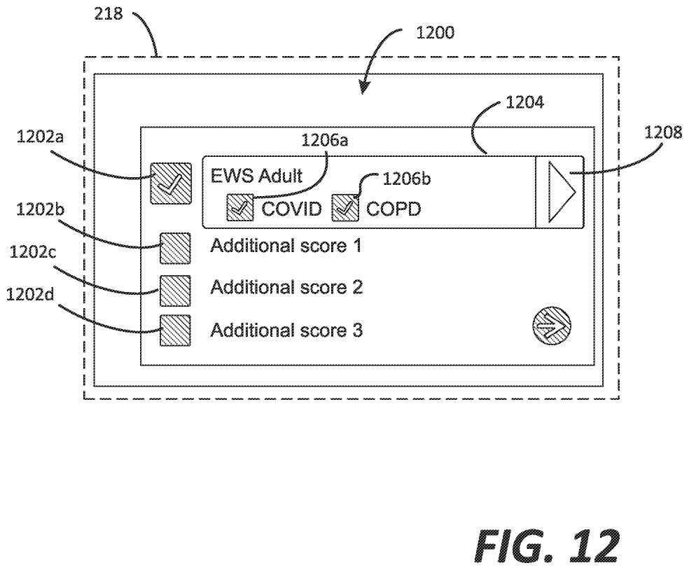

As further shown in FIG. 6, the health score 600 can include disease variants 610, which can provide one or more modifications of the health score 600 based on a disease state of a patient. For example, one or more of the physiological parameters 602, the algorithm 604, and the alarm settings 606 can be modified based on a disease state of a patient. Aspects of the disease variants 610 will be described in more detail below with reference to FIGS. 11 and 12.

FIG. 7 schematically illustrates an example of a method 700 of displaying one or more health scores for selection on the medical device 106. In some examples, the method 700 is performed by the medical device 106 before operation 306 in the method 300.

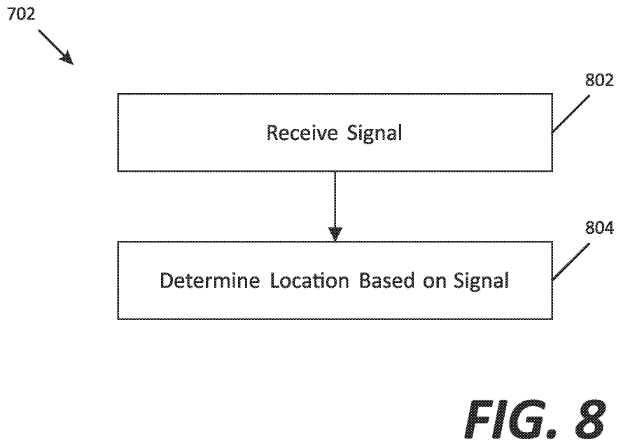

As shown in FIG. 7, the method 700 includes an operation 702 of determining a clinical setting under which the medical device 106 is operated. In some examples, the clinical setting is determined based on a location where the medical device 106 is being used within the clinical care environment 10 such as within a particular department, clinic, or area of the clinical care environment. FIG. 8, which will be described in more detail below, provides further details on how the location of the medical device 106 can be determined in operation 702.

In other examples, the clinical setting determined in operation 702 can include in addition to or as an alternative to the location of the medical device 106, a workflow in which the medical device 106 is being used. For example, operation 702 can include determining whether the medical device 106 is being used in a monitoring workflow, a spot check workflow, or a triage workflow. In some examples, the type of workflow can be determined based on the selection of the workflow that is received in operation 302 of the method 300.

In further examples, the clinical setting determined in operation 702 can include a type of patient which the medical device 106 is used to monitor and/or measure physiological parameters. This can be in addition to or as an alternative to the location and/or workflow in which the medical device 106 is being used. For example, operation 702 can include determining whether the medical device 106 is being used to monitor a pediatric patient, a geriatric patient, a patient having certain disease states, and the like. In some examples, the type of patient can be determined based on the patient identity received in operation 304 of the method 300.

Next, the method 700 includes an operation 704 of filtering a plurality of health scores. Operation 704 can include filtering the plurality of health scores based on whether the health scores are appropriate for use in the clinical setting determined in operation 702.

Next, the method 700 includes an operation 706 of displaying the filtered health scores on the display screen 218 of the medical device 106. In examples where the display screen 218 is a touchscreen, one or more of the filtered health scores can be selected by a user touching the display screen 218 of the medical device 106. In some examples, the selection of the filtered health scores is received in operation 306 in the method 300 of generating health scores on the medical device 106, which has been described above with reference to FIG. 3.

In operation 706, only filtered health scores that are appropriate for the clinical setting determined in operation 702 are displayed for selection by the user of the medical device 106. This can reduce the total number of health scores that are available for selection by the user of the medical device 106, which can improve management of the health scores on the medical device 106 especially when a large list of health scores is defined for use in the clinical care environment 10. Also, this can reduce and/or eliminate human errors because inappropriate health scores are not available for selection by a user of the medical device 106.

Additionally, the method 700 can eliminate the need to load specific health scores and protocols on the medical device 106 and/or to re-program the medical device 106 each time the medical device is moved to a different location in the clinical care environment 10 or is used in a new healthcare setting. Instead, the medical device 106 can be moved freely about the clinical care environment 10 and can be used in various healthcare settings as may be needed because the method 700 can ensure that only appropriate health scores are displayed for selection based on the context in which the medical device 106 is used in the clinical care environment 10.

FIG. 8 schematically illustrates in more detail an example of the operation 702 in the method 700, where the clinical setting is determined based on a location of the medical device 106 in the clinical care environment 10. The operation 702 can include a step 802 of receiving a signal. In some instances, the signal is generated by the tag 230 attached to the medical device 106, and the signal is received by a fixed reference point 232 positioned in a designated location in the clinical care environment 10. In another example, the signal is generated by a fixed reference point 232, and is received by the tag 230 attached to the medical device 106.

Next, the operation 702 can include a step 804 of determining a location of the medical device 106 based on the received signal. For example, the location of the medical device 106 can be determined to be the location of the fixed reference point 232. In some examples, the location of the medical device 106 is determined by the RTLS 110, which can receive an alert from the fixed reference point 232 via the network 108, and which can relay the location via the network 108 for use by the medical device 106. In other example, the medical device 106 determines its location based on the signal received from the fixed reference point 232.

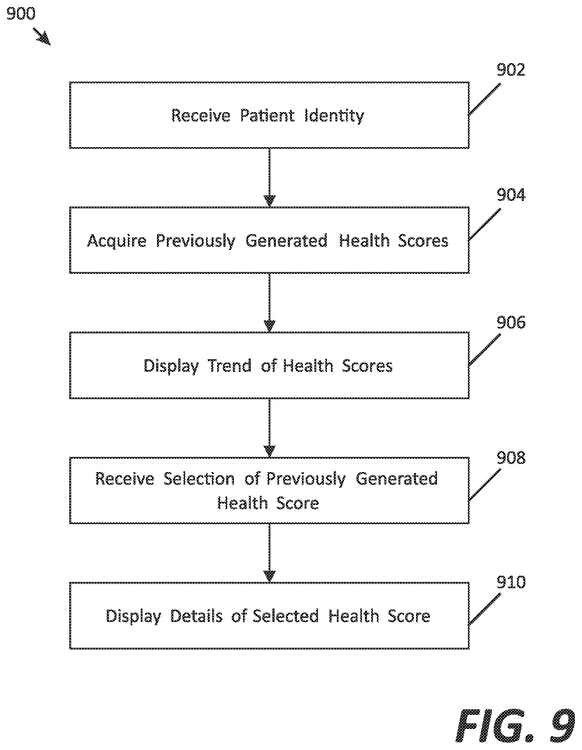

FIG. 9 schematically illustrates an example of a method 900 of displaying one or more previously generated health scores on the medical device 106. In this example embodiment, the one or more health scores are previously generated by the medical device 106 when operated by the same user or by a different user, or by one or more different medical devices. The method 900, when performed on the medical device 106, can provide a trend of the one or more health scores over a predetermined period of time, as well as detailed information on each health score included in the trend that is generally not available at bedside.

As shown in FIG. 9, the method 900 includes an operation 902 of receiving a patient identity. Operation 902 can be similar to operation 304 in the method 300, as described above. For example, operation 902 can include receiving the patient identity from a module on the medical device 106 that includes a scanner that can be used by the user of the medical device 106 to scan a barcode on a wristband or other accessory worn by the patient. Alternatively, operation 902 can include receiving a selection of the patient from a menu displayed on the display screen 218. Additional techniques for receiving the patient's identity are possible.

The method 900 includes an operation 904 of acquiring the one or more previously generated health scores. In one example embodiment, operation 904 includes the medical device 106 pulling the one or more previously generated health scores from the EMR system 102 via communications over the network 108. Alternatively, the operation 904 can include acquiring the one or more previously generated health scores from a local memory on the medical device 106.

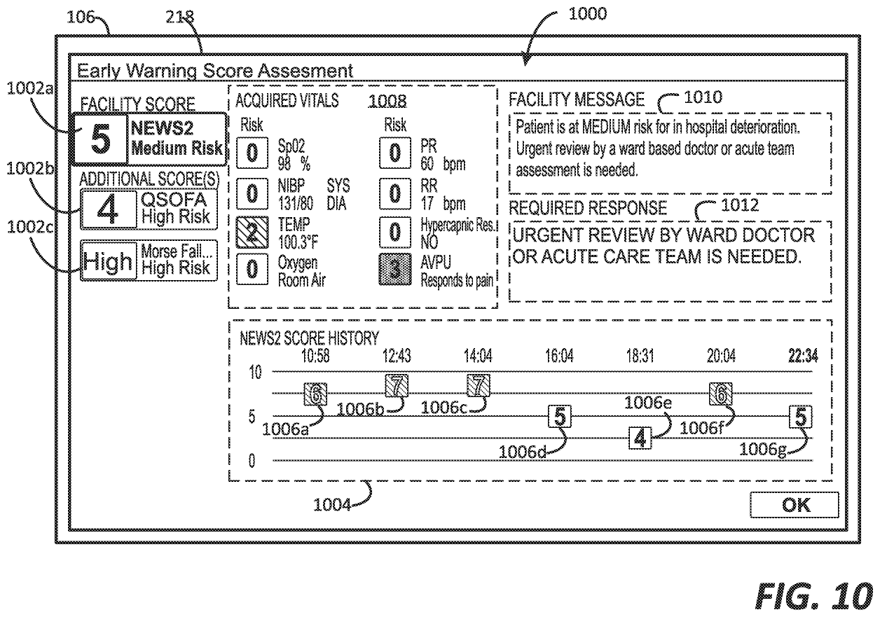

Next, the method 900 includes an operation 906 of displaying a trend of the previously generated health scores over a predetermined period of time. FIG. 10 illustrates an example of a user interface 1000 generated on the medical device 106, the user interface 1000 including a trend 1004 of the previously generated health scores 1006a-1006g. In this illustrative example, the trend 1004 displays the previously generated health scores 1006a-1006g as part of a NEWS2 score history over a predetermined period of time of approximately 12 hours. The user interface 1000 can display the trend 1004 based on a selection of a first health score 1002a (e.g., NEWS2) which includes the most recent value (e.g., “5”) for this health score. The user interface 1000 can display additional trends based on selection of additional health scores such as a second health score 1002b (e.g., QSOFA) and a third health score 1002c (e.g., “Morse Fall Scale”).

In this illustrative example, the user interface 1000 displays acquired physiological parameters 1008 that are used for calculating the most recent value (e.g., “5”) of the first health score 1002a (e.g., NEWS2). In this illustrative example, the acquired physiological parameters 1008 include an SpO2 measurement of 98% which is within a normal healthy range such that it has a parameter sub-score of “0”, a non-invasive systolic/diastolic blood pressure of 131/80 which is within a normal range such that it has a parameter sub-score of “0”, a body temperature of 100.3° C. that is elevated and has a parameter sub-score of “2”, that the patient is breathing room air instead of supplemental O₂ giving a parameter sub-score of “0”, a pulse rate of 60 beats per minute which is within a normal range such that it has a parameter sub-score of “0”, a respiration rate of 17 breaths per minute which is within a normal range such that it has a parameter sub-score of “0”, the patient is not experiencing hypercapnic respiratory failure giving a parameter sub-score of “0”, and an AVPU score of “3”. The parameter sub-scores in the acquired physiological parameters 1008 when combined together give the first health score 1002a (e.g., NEWS2) a total composite score of “5”.

Additionally, the user interface 1000 displays a facility message 1010 based on the numerical value (e.g., “5”) of the first health score 1002a. As an illustrative example, the facility message 1010 can include textual message such as “Patient is at MEDIUM risk for in-hospital deterioration. Urgent review by a ward based doctor or acute team assessment is needed.”

Additionally, the user interface 1000 can further display a required response message 1012 based on the numerical value (e.g., “5”) of the first health score 1002a. As an illustrative example, the required response message 1012 can include a textual message such as “URGENT REVIEW BY WARD DOCTOR OR ACUTE CARE TEAM IS NEEDED.”

Referring back to FIG. 9, the method 900 can include an operation 908 of receiving a selection of a previously generated health score 1006a-1006g. In examples where the display screen 218 is a touchscreen, a user of the medical device 106 can select one of the previously generated health scores 1006a-1006g displayed inside the trend 1004 by touching it.

Next, the method 900 can include an operation 910 of displaying details of the previously generated health score 1006a-1006g whose selection is received in operation 908. For example, operation 910 can include displaying the acquired physiological parameters 1008, the facility message 1010, and the required response message 1012 for the previously generated health score 1006a-1006g whose selection is received in operation 908. The method 900 when performed on the medical device 106 allows a user such as a caregiver to view an overall health score trend over time at the point of care such as the patient's bed. Also, the method 900 allows the user to view detailed information regarding previously generated health scores, such as the acquired physiological parameters 1008, the facility message 1010, and the required response message 1012 which can help the user complete a more accurate health assessment of the patient.

FIG. 11 schematically illustrates an example of a method 1100 of generating one or more health scores on the medical device 106 based on a disease state of a patient. The various health scores described above are standardized to include a standard set of physiological parameters that are allocated parameter sub-scores based on comparison to standardized ranges or values. In the example embodiment that will now be described with reference to FIGS. 11 and 12, the health scores are customizable based on a disease state of a patient such that the health scores can be used across patient populations regardless of a patient's disease state.

The method 1100 includes an operation 1102 of receiving a selection of a health score. Operation 1102 can be similar to operation 306 in the method 300 described above. Operation 1102 can include displaying the health score on the display screen 218 for selection by a user of the medical device 106. In examples where the display screen 218 is a touchscreen, the patient can touch the display screen 218 to select the health score on the medical device 106.

FIG. 12 illustrates an example of a user interface 1200 generated on the display screen 218 of the medical device 106. The user interface 1200 includes one or more health scores 1202 that can be selected by the user of the medical device 106. In accordance with the examples described above, the user can select a single health score, or can select two or more health scores to generate multiple health scores from a single patient assessment, as described above in the method 300. In this illustrative example, a first health score 1202a is selected by the user, as indicated by an icon such as a checkmark or similar design appearing next to the health score. Additional health scores, such as a second health score 1202b, a third health score 1202c, and a fourth health score 1202d remain unselected in the illustrative example shown in FIG. 12.

Referring now back to FIG. 11, the method 1100 next includes an operation 1104 of determining whether the patient has a disease state that could influence the health score selected in operation 1102. For example, temporary or chronic disease states can affect and/or influence certain physiological parameters, and thereby influence the overall health score. As an illustrative example, chronic obstructive pulmonary disease (COPD) effects a patient's ability to breath, and can thereby influence physiological parameters such as respiration rate and blood oxygen saturation. The influence on these physiological parameters can cause elevated individual parameter sub-scores, and thereby increase the overall health score which can trigger alarms and medical protocols even though the patient is in a stable condition for their given disease state.

In one example embodiment, operation 1104 can include automatically determining whether the patient has a disease state such as by having the medical device 106 pull the patient's electronic medical record from the EMR system 102 via communications over the network 108, and then review the electronic medical record to determine whether the patient has any disease diagnosis that could influence the health score selected in operation 1102.

In another example embodiment, operation 1104 can include providing a user of the medical device 106 the ability to flag a patient for assessment with a disease specific health score variant. This is shown in the illustrative example provided in FIG. 12, where each of the health scores 1202 when selected by the user of the medical device 106 can result in the medical device 106 displaying a disease variant box 1204 that includes one or more disease variants 1206. In this illustrative example, the disease variant box 1204 for the first health score 1202a includes a first disease variant 1206a (e.g., “COVID”) and a second disease variant 1206b (e.g., “COPD”) that can be selected by the user to alter and/or customize the first health score 1202a based on these diseases. Additional types of disease variants are contemplated such that the disease variants 1206a, 1206b shown in FIG. 12 are provided by way of illustrative example. In some examples, the disease variant box 1204 can include more than two disease variants 1206, or can include only a single disease variant. The disease variants 1206 can be defined for some of the health scores 1202, but not necessarily all of the health scores 1202 such that in some examples, selection of a health score 1202 does not result in the display of the disease variant box 1204.

Referring now back to FIG. 11, when the patient is determined as not having a disease state that can potentially influence the one or more health scores selected in operation 1102 (i.e., “No” in operation 1104), the method 1100 proceeds to an operation 1106 of generating the one or more health scores as standardized health scores using physiological parameter measurements and clinical observations obtained using the various modules of the medical device 106. In some instances, operation 1106 can include any one or more of the operations 308-316 included in the method 300, described above with reference to FIG. 3.

When the patient is determined as having one or more disease states that can potentially influence the one or more health scores selected in operation 1102 (i.e., “Yes” in operation 1104), the method 1100 proceeds to an operation 1108 of modifying and/or customizing the one or more health scores selected in operation 1102 based on the one or more disease states. For example, operation 1108 can include modifying any one or more of the physiological parameters 602, algorithm 604, alarm settings 606, and clinical settings 608 that are defined for the health score 600. Such modifications can cause certain physiological parameters to be ignored or can include deemphasizing certain physiological parameters and/or emphasizing certain physiological parameters over others. Such modification can further include adjusting alarm limits to reduce false alarms and alarm fatigue. In some examples, the health score that is modified or customized based on the one or more disease states is associated with the disease variant 610, and is saved on a memory of the medical device 106 or elsewhere. Thereafter, the method 1100 can proceed to operation 1106 of generating the one or more health scores as modified and/or customized health scores using physiological parameter measurements and clinical observations obtained using the various modules of the medical device 106.

As further shown in FIG. 12, the user interface 1200 can further include an icon 1208 that when selected by the user of the medical device can display additional information such as how the selected disease variants modify the selected health score, such as whether they ignore certain physiological parameters or use weights and other coefficients to deemphasize certain physiological parameters or to emphasize certain physiological parameters over others.

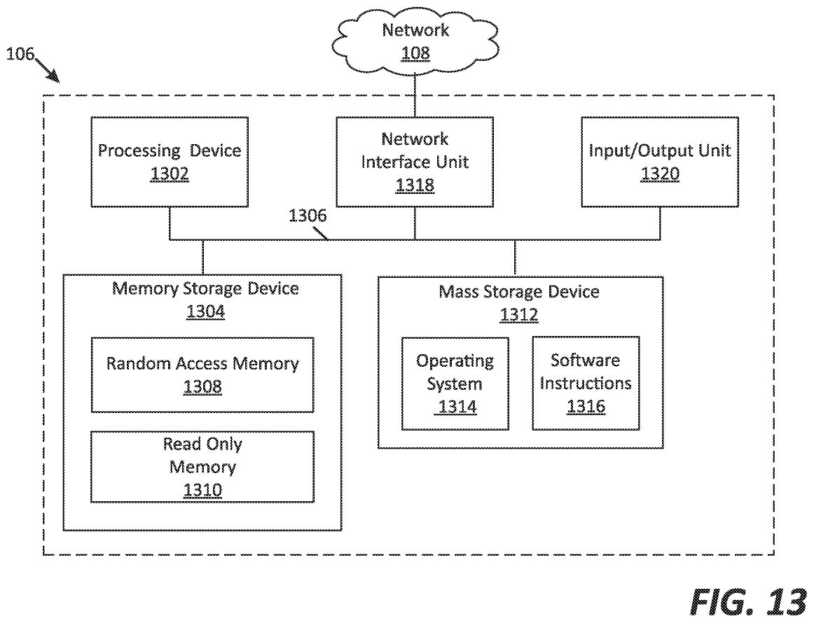

FIG. 13 schematically illustrates an example of the medical device 106 that can be used to implement aspects of the present disclosure. As shown in the example provided in FIG. 13, the medical device 106 includes a processing device 1302, a memory storage device 1304, and a system bus 1306 that couples the memory storage device 1304 to the processing device 1302. The processing device 1302 is an example of a central processing unit (CPU).

As shown in FIG. 13, the memory storage device 1304 can include a random-access memory (“RAM”) 1308 and a read-only memory (“ROM”) 1310. Basic input and output logic having basic routines that help to transfer information between elements within the medical device 106, such as during startup, can be stored in the ROM 1310.

The medical device 106 can also include a mass storage device 1312 that can include an operating system 1314 and store software instructions 1316 and data. The mass storage device 1312 is connected to the processing device 1302 through the system bus 1306. The mass storage device 1312 and associated computer-readable data storage media provide non-volatile, non-transitory storage for the medical device 106.

Although the description of computer-readable data storage media contained herein refers to the mass storage device 1312, it should be appreciated by those skilled in the art that computer-readable data storage media can be any available non-transitory, physical device or article of manufacture from which the medical device 106 can read data and/or instructions. The computer-readable storage media can be comprised of entirely non-transitory media. The mass storage device 1312 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The medical device 106 operates in a networked environment using logical connections to other medical devices and other electronic devices through the network 108. The medical device 106 connects to the network 108 through a network interface unit 1318 connected to the system bus 1306. The network interface unit 1318 can also connect to additional types of communications networks and devices, including through Bluetooth, Wi-Fi, and cellular telecommunications networks including 4G and 5G networks. The network interface unit 1318 may also connect the medical device 106 to additional networks, systems, and devices such as the EMR system 102, a digital health gateway, and other clinical resource centers.

The medical device 106 includes an input/output unit 1320 for receiving and processing inputs and outputs from a number of peripheral devices. Examples of peripheral devices can include, without limitation, a temperature probe, blood pressure cuffs, ECG or EKG leads, a clip for measuring blood oxygen saturation and pulse, and other peripheral devices.

The mass storage device 1312 and the RAM 1308 can store software instructions and data. The software instructions can include an operating system 1314 suitable for controlling the operation of the medical device 106. The mass storage device 1312 and/or the RAM 1308 can also store software instructions 1316, which when executed by the processing device 1302, cause the device to provide the functionality of the medical device 106 discussed herein.

The various embodiments described above are provided by way of illustration only and should not be construed to be limiting in any way. Various modifications can be made to the embodiments described above without departing from the true spirit and scope of the disclosure.

What is claimed is:

1. A medical device for assessing a health status of a patient, the medical device comprising:
at least one processing device; and
a memory device storing instructions which, when executed by the at least one processing device, cause the at least one processing device to:
receive a selection of two or more health scores;
obtain physiological parameter measurements from one or more sensor modules based on the selection of the two or more health scores;
generate the two or more health scores using the physiological parameter measurements received from the one or more sensor modules, the two or more health scores being generated based on a single workflow for assessing the patient; and
display the two or more health scores on a display screen.

2. The medical device of claim 1, wherein the instructions further cause the at least one processing device to:
store the two or more health scores in an electronic medical record.

3. The medical device of claim 1, wherein the instructions further cause the at least one processing device to:
determine a sequence of tasks for collecting the physiological parameter measurements based on the selection of the two or more health scores.

4. The medical device of claim 1, wherein the instructions further cause the at least one processing device to:
display the physiological parameter measurements and associated parameter sub-scores used for generating the two or more health scores.

5. The medical device of claim 1, wherein the instructions further cause the at least one processing device to:
determine a clinical setting under which the medical device is operated;
filter a plurality of health scores based the clinical setting; and
display a subset of health scores from the plurality of health scores for selection, the subset of health scores including health scores determined appropriate for the clinical setting.

6. The medical device of claim 5, wherein the clinical setting is determined based on a location of the medical device within a clinical care environment.

7. The medical device of claim 6, further comprising:
a tag attached to the medical device, the tag being configured to communicate with fixed reference points positioned in designated locations throughout the clinical care environment for determining the location of the medical device within the clinical care environment.

8. The medical device of claim 1, wherein the instructions further cause the at least one processing device to:
display individual progress indications for each of the two or more health scores.

9. The medical device of claim 1, wherein the instructions further cause the at least one processing device to:
acquire previously generated health scores;
display a trend based on the previously generated health scores;
receive a selection of a particular health score within the trend; and
display the physiological parameter measurements and associated parameter sub-scores used for generating the particular health score selected within the trend.

10. The medical device of claim 1, wherein the instructions further cause the at least one processing device to:
determine whether the patient has a disease state; and
customize the two or more health scores based on the disease state.

11. A medical device for assessing a health status of a patient, the medical device comprising:
at least one processing device; and
a memory device storing instructions which, when executed by the at least one processing device, cause the at least one processing device to:
determine a clinical setting of the medical device;
filter a plurality of health scores based the clinical setting;
display a subset of health scores from the plurality of health scores, the subset of health scores including health scores determined appropriate for the clinical setting;
receive a selection of two or more health scores from the subset of health scores;
obtain physiological parameter measurements based on the selection of the two or more health scores;
generate the two or more health scores using the physiological parameter measurements; and
store the two or more health scores in an electronic medical record.

12. The medical device of claim 11, wherein the instructions further cause the at least one processing device to:
determine the clinical setting based on a location of the medical device within a clinical care environment.

13. The medical device of claim 12, further comprising:
a tag attached to the medical device, and wherein the location of the medical device is determined by the tag communicating with fixed reference points positioned in designated locations throughout the clinical care environment.

14. The medical device of claim 11, wherein the two or more health scores are simultaneously generated based on a single workflow for assessing the patient.

15. The medical device of claim 11, wherein the instructions further cause the at least one processing device to:
determine a sequence of tasks for collecting the physiological parameter measurements based on the selection of the two or more health scores.

16. The medical device of claim 11, wherein the instructions further cause the at least one processing device to:
display the physiological parameter measurements and associated parameter sub-scores used for generating each of the two or more health scores.

17. The medical device of claim 11, wherein the instructions further cause the at least one processing device to:
acquire previously generated health scores;
display a trend based on the previously generated health scores;
receive a selection of a particular health score within the trend; and
display the one or more physiological parameter measurements and associated parameter sub-scores used for generating the particular health score selected within the trend.

18. The medical device of claim 11, wherein the instructions further cause the at least one processing device to:
determine whether the patient has a disease state; and
customize the two or more health scores based on the disease state.

19. The medical device of claim 11, wherein the instructions further cause the at least one processing device to:
determine the clinical setting based on a selection of a workflow or an identification of the patient.

20. A method of assessing a patient in a clinical care environment, the method comprising:
determining a clinical setting in which a medical device is being operated;
filtering a plurality of health scores based the clinical setting;
displaying a subset of health scores from the plurality of health scores, the subset of health scores including health scores determined appropriate for the clinical setting;
receiving a selection of two or more health scores from the subset of health scores;
obtaining physiological parameter measurements based on the selection of the two or more health scores;
generating the two or more health scores using the physiological parameter measurements; and
storing the two or more health scores in an electronic medical record.
